(19) United States
(12) Patent Application Publication
(10) Pub. No.: US 2022/0249305 A1
(43) Pub. Date: Aug. 11, 2022

(54) Clean Diaper and Wipe Dispensing Cabinet

(71) Applicant: Sharon Norman, Smiths Station, AL (US)

(72) Inventor: Sharon Norman, Smiths Station, AL (US)

(21) Appl. No.: 17/666,033

(22) Filed: Feb. 7, 2022

Related U.S. Application Data
(60) Provisional application No. 63/146,267, filed on Feb. 5, 2021.

(51) Int. Cl. A61F 15/00; A47K 10/42

(52) U.S. Cl. CPC A61F 15/001; A47K 10/421

(57) ABSTRACT

A diaper and wipe dispensing cabinet is provided. The device includes a housing centrally disposed on a panel. The housing further includes a front door. The front door provides access to an interior volume of the housing in which diapers are stored. A lower end of the interior volume includes a spring loaded platform that biases a spring upwardly, thereby forcing stored diapers upwardly within the interior volume toward a wide upper region of an aperture disposed on the front door. The wide upper region allows for easy retrieval a diaper when the front door is secured to the housing via a latch. An upper portion of the housing further includes a plurality of members extending upwardly above the housing defining a recessed area. The panel further includes a plurality of shelves for additional storage and at least one hook to attach the device to a support structure.

### CROSS REFERENCE TO RELATED APPLICATIONS

This application claims the benefit of U.S. Provisional Application No. 63/146,267 filed on Feb. 5, 2021. The above identified patent application is herein incorporated by reference in its entirety to provide continuity of disclosure.

### BACKGROUND OF THE INVENTION

The present invention relates to a dispensing cabinet. More specifically, the present invention provides a dispensing cabinet that has a housing for storing and dispensing diapers which is removable from a panel that includes at least one support hook and a plurality of shelving for additional storage.

Caring for an infant child takes a lot of time and effort. One of the most important tasks that requires constant monitoring by the parent or guardian is caring for the child's diaper. When the infant child defecates in their diaper, it is important for the adult caring for the child to quickly remove the soiled diaper and clean the child. A failure to swiftly care for a soiled diaper may leave the child susceptible to suffering a rash or other associated illnesses that spawn for the close contact with the feces. Typically, the process of changing an infant's diaper needs to be done efficiently and quickly due to the difficulty of having the infant remaining still when on a changing table. Moreover, the adult must maintain visual contact on the child when they are on the changing table to make sure nothing harmful happens to the infant or that the infant reaches for any object. This makes it difficult for the adult to search for the necessary supplies in the middle of the changing process. Instead, the adult needs all the necessary supplies easily accessible and in a location that allows them to maintain their visual on the child. Typically, adults will store all the necessary supplies for changing a diaper within the same device.

Many of the currently available devices for storing diapers, wipes, and other necessities for infant children lack organization. Typically, the adult must search within the interior of the device to find the specific supply that they require. For example, some individuals utilize diaper bags. The diaper bags tend to be crammed with supplies and backup clothing, making it hard to find a desired item, such as a diaper of a particular size, quickly. These diaper bags may lack the necessary organizational compartments that allow a user to easily locate and remove the necessary supplies without having to momentarily divert their attention away from the child.

Moreover, adults tend to leave the supplies for taking care of an infant on shelves or counters which are approximate to the surface in which the child's diaper is changed. Often, the supplies will remain in the packaging and the adult will retrieve the item from within the packaging when needed. However, when needing to retrieve an item from the packaging the individual can struggle with removing it from the packaging. This can lead to the individual becoming overly frustrated during the changing process. Items that are easily dispensed from containers reduces the amount of frustration the individual experiences when changing the diaper of the infant and will decrease the amount of time needed to efficiently change the child's diaper.

Therefore, there is a defined need amongst the known prior art references for a dispensing cabinet that comprises a housing for storing and dispending diapers and a panel that includes at least one support hook and a plurality of shelves

### SUMMARY OF THE INVENTION

In view of the foregoing disadvantages inherent in the known types of dispensing cabinets, the present invention includes a dispensing cabinet comprising a housing that dispenses diapers and may be coupled to a panel that includes at least one support hook for connecting the device to support objects and a plurality of shelving for additional storage.

It is therefore an object of the present invention to provide a housing that dispenses diapers. The housing includes an interior volume that may house a plurality of diapers. Depending on the size of the diapers, the user may stack the diapers singularly or in pairs. Diapers will be stacked upon a spring loaded platform. The spring loaded platform is biased upwardly within the interior volume of the housing. In this way, the diapers will be positioned towards the upper section of the housing. The upper section of the housing includes a wide opening that allows a user to remove a diaper from therein without having to open a front door of the housing. As diapers begin to be removed from the housing, the spring loaded platform will push the remaining diapers upward towards the wide opening to allow for easy access to the diapers for any subsequent changing that is required.

It is therefore another object of the present invention to provide a panel which connects with the housing. The panel includes at least one retractable hook that allows the panel to be connected to a changing table, a horizontal crossbar, or a similar hanging support structure. Connecting the device with one of those objects via the retractable hook allows the device to be placed in a location which is convenient for the user while they are changing the infant's diaper. Moreover, the panel includes a plurality of shelves. The plurality of shelves provides space for additional items to be stored and organized. In one embodiment, the position of the shelves on the panel may be adjusted depending on the size of the item that the user places thereon. This includes a recessed area disposed on the upper end of the housing. The recessed area is defined by a plurality of members that extend upwardly from the housing. A user may place a wipe dispensing container within the recessed area for quick access to wipes when needed during the diaper changing process.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a perspective view of an embodiment of the diaper and wipe dispensing cabinet.

FIG. 1B shows a partially exploded view of an embodiment of the diaper and wipe dispensing cabinet.

FIG. 2A shows a side view of an embodiment of the diaper and wipe dispensing cabinet.

FIG. 2B shows a side view of an embodiment of the diaper and wipe dispensing cabinet.

FIG. 3 shows a front view of an embodiment of the diaper and wipe dispensing cabinet.

### DETAILED DESCRIPTION OF THE INVENTION

Reference is made herein to the attached drawings. Like reference numerals are used throughout the drawings to depict like or similar elements of the diaper and wipe dispensing cabinet. The figures are intended for representative purposes only and should not be limiting in any respect.

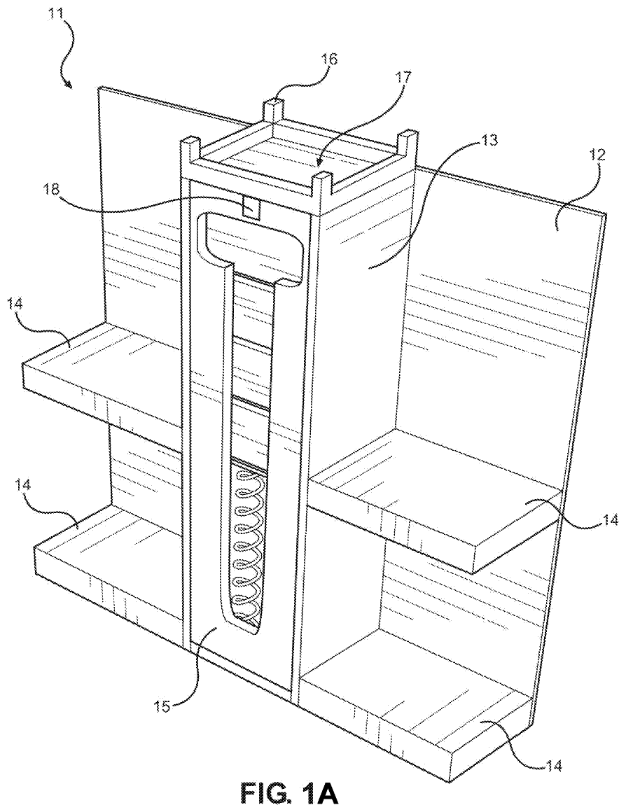

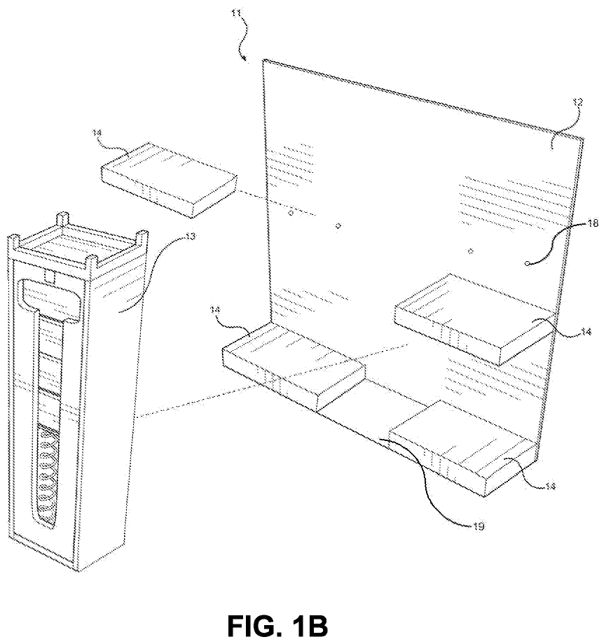

FIG. 1A shows a perspective view of an embodiment of a diaper and wipe dispensing cabinet. The diaper and wipe dispensing cabinet 11 comprises a panel 12 and a housing 13. The panel 12 includes a front face and a rear face. The housing 13 is centrally disposed to the front face of the panel 12. In one embodiment, the housing 13 is removable from the panel 12. The housing 13 is removably securable to the panel 12 via a friction fit. The housing 12 includes an upper end and a lower end. A front door 15 is disposed on the housing 13. A latch 18 disposed towards the upper end of the housing 12 secures the front door 15 in a closed position. The latch 18 may be selectively actuated by a user to disengage the front door 15 from the housing 13. While the front door 15 is disengaged from the housing 13, the front door 15 is in an open position. In an open position, a user may access an interior volume of the housing 13 for restocking of supplies therein.

A plurality of members 16 extends upwardly from above the housing 13. The plurality of members 16 define a recessed area 17. The recessed area 17 permits a user to place a wipe dispensing container or similar items therein. While within the recessed area 17, the user will have quick access to the wipes within the wipe dispensing container. The plurality of members 16 will prevent the item placed within the recessed area 17 from falling off the housing 13. A plurality of shelves 14 is disposed on the front face of the panel 12. The plurality of shelves 14 are placed on either side of the housing 13. The plurality of shelves 14 provides the user with storage space for additional items. In one embodiment, the position of the plurality of shelves 14 along the panel 12 is adjustable to accommodate for the items thereon.

FIG. 1B shows a partially exploded view of an embodiment of the diaper and wipe dispensing cabinet. In one embodiment, the housing 13 maybe disconnect from the panel 12. In the shown embodiment, the housing 13 be placed upon a surface 19. The surface 19 is disposed on the panel 12. When placed upon the surface 19, the housing 13 is secured in place via a friction fit. In some embodiment, other securing devices may be utilized to further secure the housing to the panel 12. Moreover, the plurality of shelves 14 connect to the panel 12 via at least one shelf fastener 18. The at least one shelf fastener 18 is disposed along the panel 12. The user may select a shelf fastener to secure the plurality of shelves 14 thereto along the panel 12 for a desired a configuration of the plurality of shelves 14. This permits the user to adjust the plurality of shelves 14 along the panel 12 to accommodate for items of varying sizes on to the diaper and wipe dispensing cabinet 11.

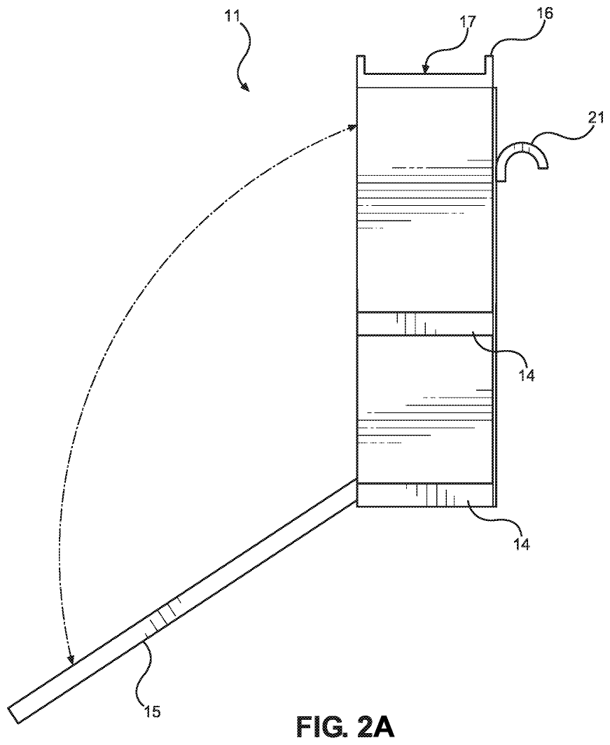

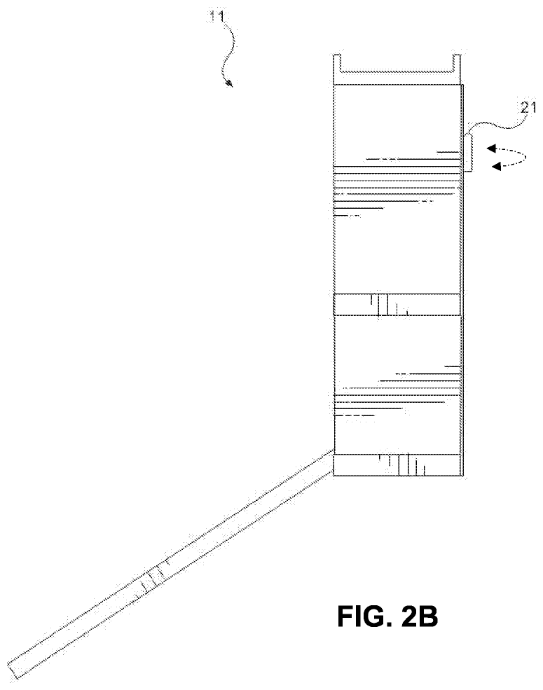

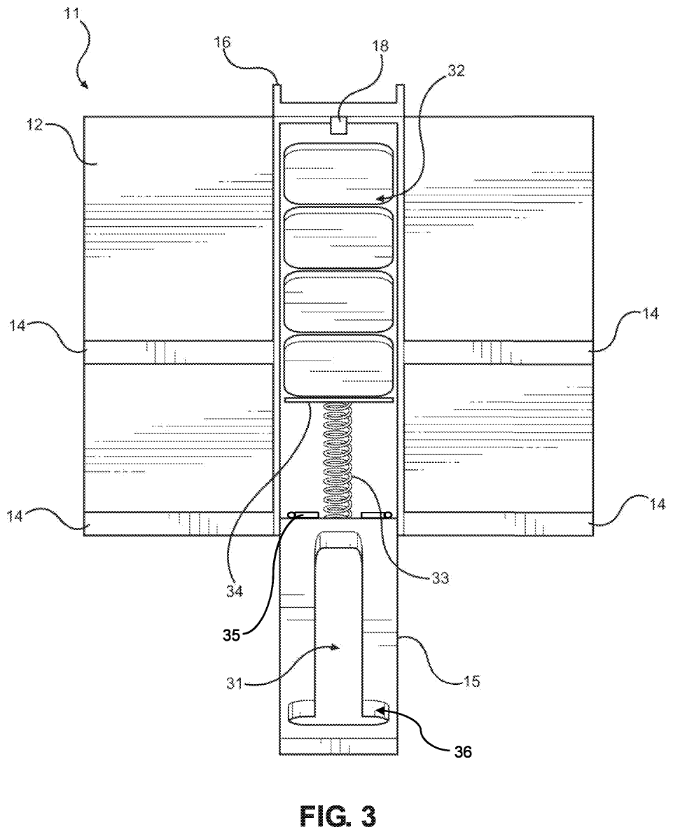

FIGS. 2A and 2B show side views of an embodiment of a diaper and wipe dispensing cabinet. The diaper and wipe dispensing cabinet 11 includes the housing with the front door 15. The latch secures the front door 15 to the housing. When the latch is engaged, the front door 15 is released. As the front door 15 is released, the front door 15 will rotate about at least one hinge (as shown in FIG. 3, 35). In one embodiment, the at least one hinge is located towards the lower end of the housing. The front door 15 provides access to an interior volume of the housing when the front door 15 is not secured via the latch. When the interior volume of the housing is accessible, a user may place the desired items therein. The user may encase the objects within the housing by lifting the front door 15 about the at least one hinge and secure the position of the front door 15 by engaging the front door 15 via the latch.

At least one hook 21 is disposed on the rear face of the panel. In one embodiment, the at least one hook 21 is retractable. In such embodiment, the at least one hook 21 may be positioned in a usage configuration or a storage configuration. In the storage configuration the at least one hook 21 is flush with the rear face of the panel, in that the at least one hook 21 is parallel with the panel. The at least one hook 21 may rotate 90-degrees away from the panel to be in the usage configuration. In the usage configuration, the at least one hook 21 extends outwardly from the panel, in that the at least one hook 21 is perpendicular with the panel. When in the usage configuration, the at least one hook 21 allows the diaper and wipe dispensing cabinet 11 to be secured to a changing table, a horizontal crossbar, or a similar hanging support structure. Securing the diaper and wipe dispensing cabinet 11 to said support structures allows the user to position the device in a location that is convenient and promotes quick accessibility to the items stored thereon.

When the diaper and wipe dispensing cabinet 11 is secured to a changing table, a horizontal crossbar, or a similar hanging support structure the user may still place objects on the plurality of shelves 14. The plurality of shelves 14 provides a space along the device for the user to store additional supply items. Moreover, the recessed area 17, which is defined by the plurality of members 16, may also provide storage space for a wipe dispensing container. While placed within the recessed area 17, wipes within the wipe dispensing container may be easily accessible for the user when they are performing a changing of an infant's diaper or any similar task.

FIG. 3 shows a front view of an embodiment of the diaper and wipe dispensing cabinet. The diaper and wipe dispensing cabinet 11 provides a housing connected to a panel 12. The housing includes an interior volume 32 which can store items, such as diapers. The interior volume 32 of the housing comprises a spring loaded platform 34. The spring loaded platform 34 is coupled with a spring 33. The spring 33 is connected to the lower end of the housing. The spring 33 extends upwardly within the interior volume 32. The spring loaded platform 34 rests above the spring 33. The spring loaded platform 34 is biased upwards within the interior volume 32. The items placed upon the spring loaded platform 34 displace the spring 33 which lowers the spring loaded platform 34 within the interior volume 32. In one embodiment, the housing is sized and shaped to store up to two sleeves of diapers side by side therein. Moreover, the diaper and wipe dispensing cabinet 11 includes a plurality of shelves 14 which are disposed on the front face of the panel 12 on both sides of the housing and a plurality of members 16 extending upwardly from the housing to define the recessed area for additional storage options.

An aperture 31 extends through the front door 15. The aperture 31 permits a user to see the contents within the housing, when the front door 15 is in a closed position by engaging with the latch 18. The user may load more items within the interior volume 32 when the front door 15 is in an open position and disengaged with the latch 18. In the shown embodiment, the aperture 31 further includes a wide upper region 36. The wide upper region 36 of the aperture 31 permits a user to retrieve an item from the interior volume 32 when the front door 15 is in the closed position. In the shown embodiment, the aperture 31 includes a longitudinal aperture. The longitudinal aperture extends along length of front door 15 for the user to see the remaining number of items within the interior volume 32. Items placed within the interior volume 32 and upon the spring loaded platform 34, compresses the spring 33. Compressing the spring 33 will displace the spring loaded platform 34. The bias of the spring loaded platform 34 will position the items towards the wide upper region 36 of the aperture 31 for easy removal from the housing. As items are removed from the interior volume 32, the spring loaded platform 34 is displaced. The displacement of the spring loaded platform 34 expands the spring 33 which raises the spring loaded platform 34 within the interior volume 32 and place the stored items upwardly towards the wide upper region 36 of the aperture 31 for easy retrieval by the user for the next use. In some embodiments, a lip is disposed on an interior face of the front door 13. Specifically, the lip is located around the perimeter of the wide upper region 36 and partially extends into the aperture 31. The lip prevents items within the interior volume 32 from passing through the wide upper region 36 without the action of a user. An action by the user will be able to surpass the lip by partially maneuvering the item as the item is being removed from the housing.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A dispensing cabinet, comprising:
a panel;
wherein the panel includes a front face and a rear face;
a housing centrally disposed on the front face of the panel;
whereby the housing includes an upper end, a lower end, and an interior volume;
the interior volume contains a spring loaded platform;
a plurality of shelves disposed on the front face of the panel on both sides of the housing;
a plurality of members extends upwardly from the upper end of the housing defining a recessed area;
wherein the housing further includes a front door;
an aperture disposed on the front door; and
at least one hook disposed on the rear face of the panel.

2. The dispensing cabinet of claim 1, wherein the front door is attached to the housing via at least one hinge, whereby the at least one hinge is disposed on the lower end of the housing.

3. The dispensing cabinet of claim 1, wherein the front door is secured to the housing via a latch, whereby the latch is disposed on the upper end of the housing.

4. The dispensing cabinet of claim 1, wherein the spring loaded platform is placed within the interior volume towards the lower end of the housing.

5. The dispensing cabinet of claim 1, wherein the spring loaded platform is biased upwards within the interior volume.

6. The dispensing cabinet of claim 1, wherein the aperture includes a wide upper region.

7. The dispensing cabinet of claim 1, wherein the aperture includes a longitudinal aperture extending along length of front door.

8. The dispensing cabinet of claim 1, wherein the plurality of shelves is adjustable along the front face of the panel.

9. The dispensing cabinet of claim 8, wherein the plurality of shelves may connect to the front face of the panel via at least one shelf fastener.

10. The dispensing cabinet of claim 1, wherein the housing is removable from the front face of the panel.

11. The dispensing cabinet of claim 1, wherein the at least one hook is retractable; whereby the at least one hook rotates about the rear face of the panel between a usable configuration and a storage configuration.

12. The dispensing cabinet of claim 11, wherein the at least one hook in the storage configuration when the at least one hook is flush and parallel with the rear face of the panel.

13. The dispensing cabinet of claim 11, wherein the at least one hook in the usable configuration when the at least one hook extends perpendicularly outward from the rear face of the panel.

14. The dispensing cabinet of claim 1, wherein the recessed area disposed on the upper end of the housing is rectangular in shape.
